(19) United States
(12) Patent Application Publication
(10) Pub. No.: US 2006/0200180 A1
(43) Pub. Date: Sep. 7, 2006

(54) DEVICE FOR REMOVING DISEASED SURFACE TISSUES

(76) Inventor: Yi-Chang Lee, Taipei (TW)

Correspondence Address: JOE CHRISTIE PRESIDENT;GALVANIX CORPORATION, 901 MOPAC EXPRESSWAY SOUTH, BARTON OAKS PLAZA TWO SUITE 515, AUSTIN, TX, 78746, US

(21) Appl. No.: 11/164,401

(22) Filed: Nov. 21, 2005

Related U.S. Application Data
(63) Continuation-in-part of application No. 10/388,844, filed on Mar. 14, 2003, now abandoned.

(51) Int. Cl. A61B 17/32

(52) U.S. Cl. 606/169

(57) ABSTRACT

A device is designed for scraping off the diseased part at the surface of soft tissues, particularly for removing diseased tissues deep in an ear canal. The device mainly includes a handle internally provided with a vibrating motor having an eccentric wheel mounted on a rotary shaft of the motor, and a plurality of batteries; and a tubular scraper having two open ends, a front end of which is a scraper tip having a toothed front edge, and a rear end of which is connected to a front end of the handle and communicates with a suction hose. When the motor is actuated, the handle and the scraper are vibrated for the scraper tip to scrape and exfoliate diseased tissues while the suction hose synchronously sucks out effused blood and exfoliated tissues, so that the treated area is clean for easy observation during an operation.

### FIELD OF THE INVENTION

This application is a continuation in part of U.S. patent application Ser. No. 10/388,844 filed on Mar. 14, 2003.

The present invention relates to a device for removing diseased surface tissues adapted to scrape off diseased part on skin or other soft tissue. By vibrating a scraper of the device to scrape off the diseased surface tissues and synchronously sucking out the scraped matters, the area undergoing operation can be efficiently cleaned for easy observation during the operation.

### BACKGROUND OF THE INVENTION

When an area on human body has diseased tissues due to inflammation, suppuration, polyp, necrosis, etc., it is usually necessary to clear the diseased tissues from the area so as to provide a good re-growth environment for any wound in the area. Most existing ways of removing diseased tissues involve the use of knife, lancet, scissors, forceps, clamp, etc. An operation for removing the diseased tissues from a wound becomes particularly difficult because a surgeon performing the operation normally has to hold one of many different instruments with one hand while holding an auxiliary suction tube with another hand. Taking an operation for a hemorrhagic disorder deep in the ear canal as an example, since it is precisely performed within an extremely narrow space with the help of a microscope, the use of traditional surgical instruments would usually encounters with difficulties. Particularly, any small amount of hemorrhage at the wound would prevent the doctor from clearly observing the wound to largely reduce the accuracy and efficiency of the operation.

When there are diseased surface tissues on a certain area of the human body, it is also necessary to clear the diseased surface tissues from the area before suitable medicine can be applied to the area for treatment, so that healthy skin could grow at the diseased area again.

However, the traditional knife, lancet, scissors, forceps, clamp, etc. currently used to remove diseased tissues normally have relatively simple functions and terrible appearances to not only cause a patient's fear for the treatment, but also bring great pains to the patient.

As an experienced member in the medical field, the inventor fully understands the difficulty in performing an operation in the narrow ear canal, as well as the pains and harms suffered by a patient undergoing such operation, and therefore develops a device for removing diseased surface tissues that includes a power-actuated scraper to enable a surgeon to perform a highly precise and accurate operation with reduced efforts.

### SUMMARY OF THE INVENTION

A primary object of the present invention is to provide a device for removing diseased surface tissues that can be manipulated with only one hand to quickly and effectively remove the diseased surface tissues.

Another object of the present invention is to provide a device for removing diseased surface tissues that can be advantageously used for effective operation in a narrow canal of human body.

A further object of the present invention is to provide a device for removing diseased surface tissues that combines a special scraper, suction means, and a vibration generator to enable easy and accurate scraping off of diseased surface tissues, so as to assist doctors in efficiently treating diseased surface tissues at deep and narrow areas and to alleviate the patient's pain and discomfort during the treatment.

To achieve the above and other objects, the device for removing diseased surface tissues according to the present invention mainly includes a handle and a tubular scraper. The handle is internally provided with a vibration generator for the handle to vibrate at high speed but in very minor amplitude. The tubular scraper has two open ends, a front end of which is a forward extended scraper tip. The tubular scraper is connected to a front end of the handle, such that the vibration of the handle is transmitted to the front end of the tubular scraper, enabling the scraper tip to scrape and exfoliate the diseased surface tissues from an area.

In the present invention, the scraper tip at the front end of the scraper has a toothed front edge.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a perspective view of a device for removing diseased surface tissues according to a preferred embodiment of the present invention;

FIG. 2 is a sectioned side view of FIG. 1;

FIG. 3 is a fragmentary and enlarged perspective view of a scraper tip in the preferred embodiment of the present invention;

FIG. 4 is a sectioned side view of FIG. 3; and

FIG. 5 is a side view of a device for removing diseased surface tissues according to another embodiment of the present invention.

### DETAILED DESCRIPTION OF THE PREFERRED EMBODIMENTS

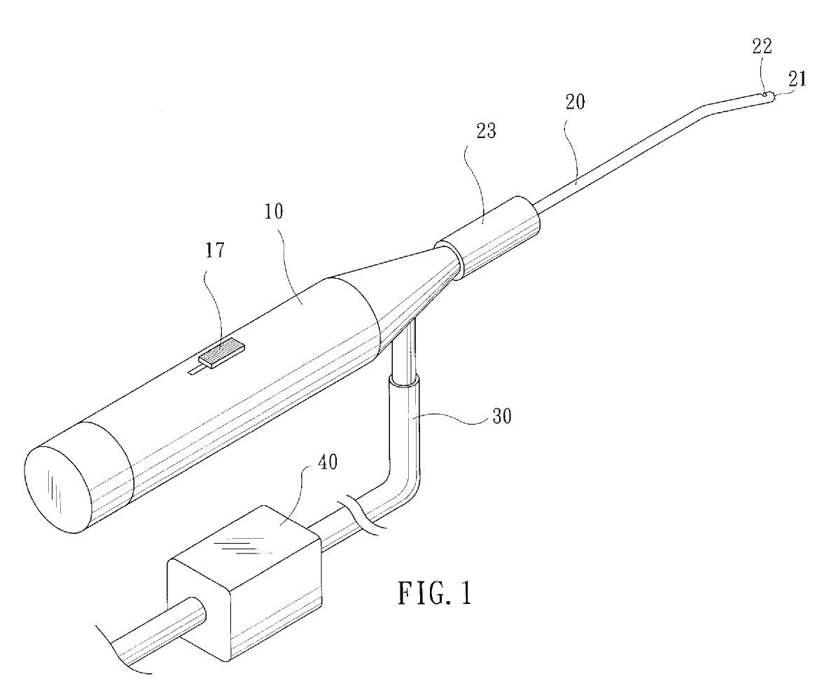

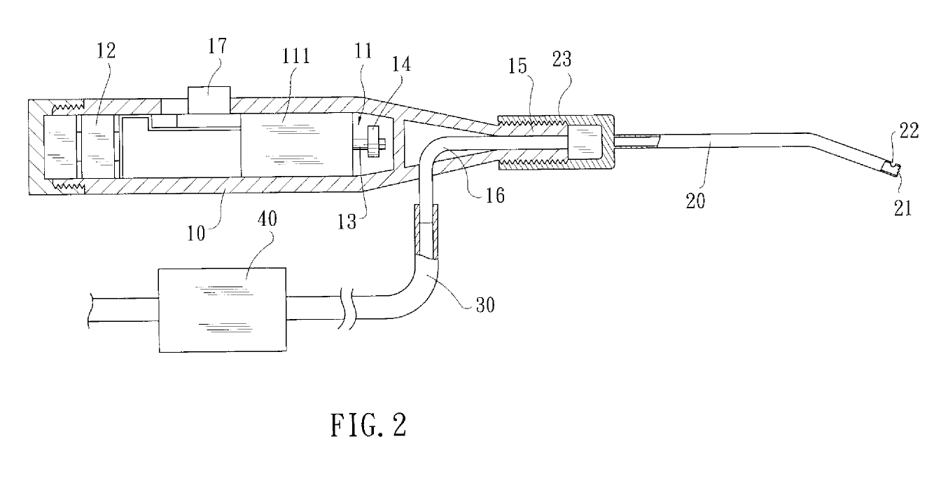

Please refer to FIGS. 1 and 2 that are perspective and sectioned side views, respectively, of a device for removing diseased surface tissues according to a preferred embodiment of the present invention. As shown, the device of the present invention mainly includes a handle 10 and a tubular scraper 20. The handle 10 is internally provided with a vibration generator 11. When the vibration generator 11 is actuated, it causes the handle to vibrate at high speed but in very minor amplitude. The tubular scraper 20 has two open ends, a front end of which is a forward extended scraper tip 21, and a rear end 23 of which is connected to an interconnecting section 15 provided at a front end of the handle 10. When the handle 10 is caused to vibrate, the vibration thereof is transmitted to the front end of the tubular scraper 20, so that the scraper tip 21 at the front end of the tubular scraper 20 is brought to vibrate at high speed in a circular motion, enabling the scraper tip 21 to scrape and exfoliate the diseased surface tissues from a diseased area.

The vibration generator 11 in the handle 10 may be a vibrating motor. That is, the vibration generator 11 may include a motor 111 and an eccentric wheel 14 connected to a rotating shaft 13 of the motor 111. When the motor 111 is actuated, it produces a high-speed vibrating effect. Batteries 12 may be provided in the handle 10 for supplying power needed to actuate the vibration generator 11. In other embodiments of the present invention, the vibration generator 11 may be driven via an externally connected power supply (not shown).

As having been mentioned above, the tubular scraper 20 is a hollow tube having two open ends, a front end of which is a scraper tip 21, and a rear open end 23 of which is connected to the interconnecting section 15 provided at the front end of the handle 10. A pipe 16 is disposed in the interconnecting section 15 with a front end of the pipe 16 connected to and communicating with a bore of the tubular scraper 20 via the rear open end 23 of the tubular scraper 20, and a rear end of the pipe 16 extended out of the interconnecting section 15 to connect to an external hose 30. The hose 30 is connected to and communicates with an air pressure pump 40. When the air-pressure pump 40 functions to suck, it sucks out matters exfoliated by the tubular scraper 20 as well as blood and body fluid effused from the patient's body to clean the area undergoing operation, so that the diseased area can be more easily observed and examined to facilitate the operation. When the air-pressure pump 40 functions to exhaust, it blows the diseased tissues exfoliated by the tubular scraper 20 away from the area undergoing operation, so that the diseased area can be more easily observed and examined. Wherein, the cleaning of the diseased area undergoing operation by exhausting air is mainly used in treating diseased tissues on skin surface.

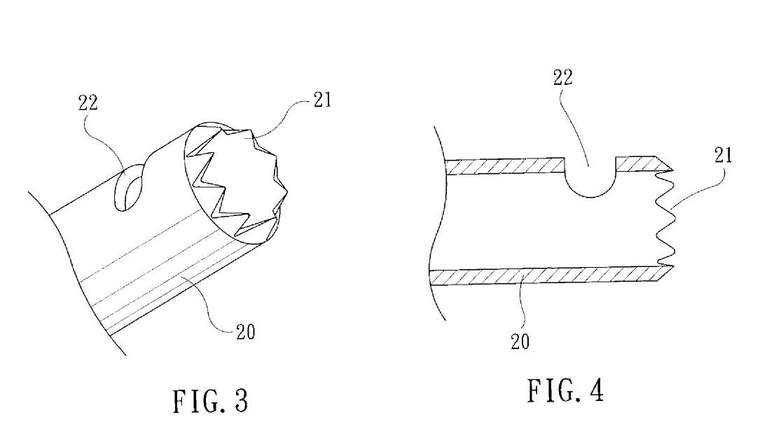

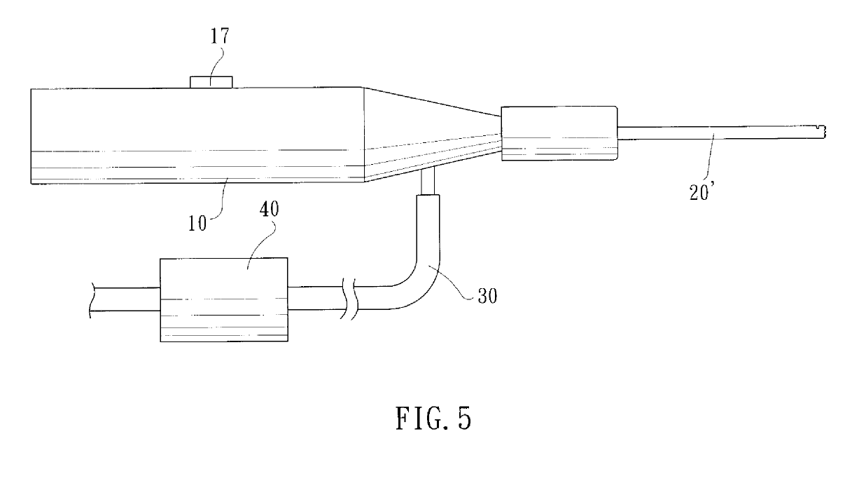

Please refer to FIGS. 3 and 4 that are fragmentary and enlarged perspective and sectioned side views, respectively, of a scrape tip 21 in the preferred embodiment of the present invention. In this embodiment, the scrape tip 21 of the tubular scraper 20 has a toothed front edge. One or two small holes 22 are provided on a sidewall of the tubular scraper 20 near the scrape tip 21 as means to balance internal pressure of the scraper 20, lest the scraper tip 21 should suck and adhere to the diseased area and become unmoved. Moreover, the small holes 22 also enable air to flow therethrough to produce a cooling effect on the scraper 20. The length of the tubular scraper 20 is one factor determining the torsion of the scraper 20. FIG. 5 shows another embodiment of the present invention having a scraper 20′ shorter than the scraper 20 in the illustrated preferred embodiment. The short scraper 20′ is more suitable for treating diseased tissues on skin surface.

When the vibration generator 11 is actuated, it causes the handle 10 and the scraper 20 to vibrate synchronously. The vibrated scraper 20 enables the scrape tip 21 to pull, cut, and exfoliate diseased tissues of a diseased area, such as gangrene, polyp, etc. Since the scraper tip 21 exfoliates the diseased tissues of the gangrene, polyp, etc. little by little, just like nibbling something, it does not forcefully pull the diseased area or injure surrounding normal tissues. To scrape polyp and gangrene having a relatively large area, a tubular scraper 20 having a scraper tip 21 with thicker or larger teeth formed at the front edge may be used to enable removal of increased amount of diseased tissues at the beginning of a medical treatment or operation. And, a scraper 20 having a scraper tip 21 with finer or smaller teeth formed at the front edge is suitable for using at a later stage during the medical treatment or operation because the finer teeth enable grinding-like scraping to thoroughly remove the remaining diseased tissues.

When the device for removing diseased surface tissues according to the present invention is used in an operation inside a patient's ear canal and/or nasal cavity, the air-pressure pump 40 communicating with the hose 30 must function to suck, so as to suck out matters exfoliated by the tubular scraper 20 as well as blood and body fluid effused from the patient's body to clean the area undergoing operation, so that the diseased area can be more easily observed and examined to facilitate the operation. The sucked matters include effused blood, body fluid, pus, and exfoliated gangrene. A doctor may manipulate the device of the present invention with only one hand during the operation, making the operation easier and more effective.

The above-described device for removing diseased surface tissues may be used in treating skin disorders and removing maculae from skin surface. That is, the device for removing diseased surface tissues may be used to exfoliate surface tissues of skin at diseased areas, allowing new skin cells to grow within several weeks. Therefore, the device may be used to replace the expensive laser maculae removal operation.

The device for removing diseased surface tissues according to the present invention may be powered with batteries 12 mounted in the handle 10, or with an external power source provided in an operation room. An on/off switch 17 is provided on the handle 10 to actuate the device.

With the above arrangements, the device for removing diseased surface tissues according to the present invention allows a doctor to manipulate it with only one hand while performing an operation. Moreover, the electrically powered device for removing diseased surface tissues enables the doctor to exfoliate the diseased tissues in an effortless manner and achieve an enhanced effect of operation while reduces pains and discomfort suffered by a patient undergoing the operation.

What is claimed is:

1. A device for removing diseased surface tissues, comprising a handle and a tubular scraper;
said handle being internally provided with a vibration generator, such that when said vibration generator is actuated, said handle is caused to vibrate at high speed but in very small amplitude; and
said tubular scraper having two open ends, a front end of which being a forward extended scraper tip;
wherein said tubular scraper is connected to a front end of said handle, such that the vibration of said handle is transmitted to the front end of said tubular scraper, enabling said scraper tip at the front end of said tubular scraper to produce high speed vibration to scrape and exfoliate diseased surface tissues from a diseased area on a patient's body.

2. The device for removing diseased surface tissues as claimed in claim 1, wherein said tubular scraper is connected to and communicable with a hose, and said hose is connected to and communicates with an air-pressure pump.

3. The device for removing diseased surface tissues as claimed in claim 1, wherein said vibration generator is a vibrating motor.

4. The device for removing diseased surface tissues as claimed in claim 3, wherein said vibrating motor includes a motor and an eccentric wheel connected to a rotating shaft of said motor.

5. The device for removing diseased surface tissues as claimed in claim 1, wherein said tubular scraper is provided on a sidewall with at least one small hole.

6. The device for removing diseased surface tissues as claimed in claim 1, wherein said scraper tip at the front end of said tubular scraper has a toothed front edge.
